(19) United States
(12) Patent Application Publication
(10) Pub. No.: US 2012/0089009 A1
(43) Pub. Date: Apr. 12, 2012

(54) METHODS AND APPARATUS TO DELIVER NANOPARTICLES TO TISSUE USING ELECTRONANOTHERAPY

(76) Inventors: Reed A. Omary, Wilmette, IL (US); Andrew C. Larson, Kildeer, IL (US); Samdeep K. Mouli, Chicago, IL (US); Aaron C. Eifler, Chicago, IL (US); Yang Guo, Chicago, IL (US)

(21) Appl. No.: 13/270,977

(22) Filed: Oct. 11, 2011

Related U.S. Application Data
(60) Provisional application No. 61/391,855, filed on Oct. 11, 2010.

(51) Int. Cl. A61B 5/055; B32B 5/16; C07H 15/24; A61N 1/30; B82Y 5/00

(52) U.S. Cl. 600/411; 536/6.4; 604/20; 428/402; 977/773

(57) ABSTRACT

Methods, systems, and apparatus are disclosed to provide delivery of nanoparticles to tissue using electro-nanotherapy or nanoablation.

### CROSS-REFERENCE TO RELATED APPLICATIONS

The present application claims the benefit of priority to U.S. Provisional Application No. 61/391,855 filed on Oct. 11, 2010, entitled “Methods and Apparatus to Deliver Nanoparticles to Tissue Using Electro-nanotherapy”, which is herein incorporated by reference in its entirety.

### FIELD OF THE DISCLOSURE

This disclosure relates generally to delivering nanoparticles to tissue, and, more particularly, to electro-nanotherapy to deliver nanoparticles to tissue using electroporation.

### BACKGROUND

In recent years, delivery of substances for treatment of tumors has been hampered by a greater permeability of surrounding cells than the tumor cells that are the target for treatment. This difference in permeability has resulted in a decrease in the effectiveness of treatment or a reliance on destroying the cell using a technique such as ablation.

### BRIEF DESCRIPTION OF THE DRAWINGS

FIG. 1 is a depiction of example magnetic resonance obtained during and after electro-nanotherapy using a rat model.

FIG. 2 illustrates an example system for cell electro-nanotherapy.

FIG. 3 shows some examples of needle electrode probes.

FIG. 4 depicts a flow diagram for an example method for performing electro-nanotherapy for a patient.

FIG. 5 is a block diagram of an example computer or other processor system that can be used to implement systems, apparatus, and methods described herein.

As used in this patent, stating that any part (e.g., a component, module, subsystem, device, control, probe, injector, imager, etc.) is in any way positioned on (e.g., positioned on, located on, disposed on, or formed on, etc.) another part, means that the referenced part is either in contact with the other part, or that the referenced part is above the other part with one or more intermediate part(s) located therebetween. Stating that any part is in contact with another part means that there is no intermediate part between the two parts.

### DETAILED DESCRIPTION

Delivery of nanoparticles (NPs) to a site of disease in a patient is a desirable modality of therapy. Nanoparticles are defined as small objects that behave as a unit with respect to its transport and properties. Nanoparticles can range in size between 1 and 100 nanometers in diameter, for example. Nanoparticles can exhibit size-dependent properties that differ from properties observed in other particles.

Nanoparticle chemistry can be used in a variety of applications including medical therapy. Superparamagnetic Iron Oxide nanoparticles (SPIOs), for example, are non-toxic and biodegradable and, when surface functionalized or coated (e.g., by covalently attaching to the surface of SPIOs) with chemotherapeutics, such as doxorubicin, are able to be absorbed or otherwise taken up by a variety of cell types. Also, chemotherapeutics attached to the SPIO nanoparticles are stable and feature a slow release profile once intracellular entry has occurred. Due to their iron content, SPIOs have a high magnetic moment and both high R2 and R2* relaxivity, and can be imaged using magnetic resonance imaging (MRI), for example, to noninvasively examine tissue uptake. Thus, SPIO nanoparticles can be used to transfect cells with chemotherapeutics and can simultaneously be used as a magnetic resonance (MR) contrast agent, permitting image-guided drug delivery.

For example, Doxorubicin is an anthracycline class antineoplastic drug used to treat a wide variety of solid and hematologic malignancies. Doxorubicin induces cytotoxicity through DNA intercalation and can be administered in chemotherapy to treat cancer in a patient.

Intravenous (IV) delivery of nanoparticles, however, is hampered by a proportionally large uptake of NPs by the reticuloendothelial system. The reticuloendothelial system (RES) is a part of a human immune system that includes phagocytic cells located in reticular connective tissue. The cells are primarily monocytes and macrophages, and they accumulate in lymph nodes and the spleen. The Kupffer cells of the liver and tissue histiocytes are also part of the RES. Cells in the RES absorb a large number of NPs injected intravenously and thereby prevent NPs from reaching desired sites in sufficient concentration.

Electroporation (abbreviated herein as EP, and also known as electropermeabilization) provides a technique to increase delivery of molecules to sites in cells and tissues that are treated. Using a series of brief electrical pulses delivered to in vivo tissues via, for example, a pair of electrodes, EP is able to make tissues more permeable to small and large molecules at the cellular level by affecting the cell plasma membrane. This permeabilization effect greatly increases an uptake or loading of NPs in the tissue that has received EP treatment and serves to “guide” NPs preferentially to the treated tissues. Using electroporation, a localized transmembrane voltage is applied to one or more points on a cell membrane. For a given electrical pulse duration and shape, a corresponding transmembrane voltage threshold is to be exceeded to manifest electroporation. Cells within areas where an electric field magnitude exceeds an electric field magnitude for electroporation provide greater permeability. Exceeding the threshold by too wide a margin can permanently damage the cells (e.g., irreversible electroporation).

Electro-nanotherapy, therefore, typically involves delivery of nanoparticles to tissues treated with electroporation. Using electro-nanotherapy (Electro-NT), the uptake of NPs in treated tissues can be greatly increased over administration of NPs alone (i.e., without electroporation treatment). Electro-nanotherapy can be used for a variety of applications including treatment of cancer, delivery of agent(s) for tissue regeneration, delivery of molecularly targeted imaging agent(s), etc. As used herein, electro-nanotherapy may also be referred to as nanoablation, for example.

Electroporation achieves disruption of cell membranes via application of an external electric field. The disruption causes otherwise low permeant or nonpermeant molecules to have increased permeability. A degree of cell membrane disruption at any given point on a cell membrane surface (M) is directly related to a transmembrane potential difference experienced at that point M, ΔVM. The transmembrane potential difference experienced at point M is related to an externally applied electric field and cell radius according to the following equation:

ΔVM=1.5×r×Eext×cos θ (Equation 1),

where r is a radius of a cell, Eext is an external electric field strength, and θ is a polar angle with respect to an electric field direction. Depending on a degree of electroporation, effects can either be reversible (e.g., a cell will return to normal with no deleterious effects after a certain length of time) or irreversible (e.g., the disruption of the cell membrane is permanent, thereby causing cell death). Electro-nanotherapy, or nanoablation, takes advantage of reversible EP to deliver NPs to cells without destroying them. However, in certain cases destruction of tissue may be desirable (e.g., in the case of solid tumor malignancies), and parameters of an externally applied electric field during an Electro-NT procedure can be adjusted to provide reversible or irreversible EP, as needed.

In certain examples, Electro-NT includes delivery of nanoparticles, either intravenously or intra-arterially, followed within a time period by electroporation at a tissue site where increased nanoparticle uptake is desired.

### BRIEF DESCRIPTION

Certain examples provide a method for electro-nanotherapy of cells at a tissue site. The method includes facilitating identification of a tissue site for electro-nanotherapy. The example method includes facilitating injection of nanoparticles at the tissue site. The example method includes enabling generation of one or more electric pulses at the tissue site to create pores in a cell membrane at the tissue site such that the nanoparticles penetrate the cell membrane.

Certain examples provide a superparamagnetic iron oxide nanoparticle treated with a chemotherapeutic to be absorbed by a cell membrane in patient tissue when the tissue is stimulated using a series of electrical pulses, the nanoparticle adapted to be imaged to permit image-guided drug delivery.

Certain examples provide a system for electro-nanotherapy of cells at a tissue site in a patient. The example system includes a nanoparticle injector to facilitate injection of nanoparticles treated with a chemotherapeutic at an identified tissue site. The example system includes a controller to enable generation of one or more electrical pulses at the tissue site to create pores in a cell membrane at the tissue site such that the nanoparticles penetrate the cell membrane.

### Examples

In an example, Electro-NT was applied to a rat model of hepatocellular carcinoma (HCC). First, the animal model was created by injecting a suspension of N1S1 cells (e.g., a rat hepatoma) into the extracorporeally exposed liver of two living rats. Rat livers were exposed using a surgical mini-laparotomy procedure and aseptically closed following cell injection. The rats survived for fourteen (14) days to allow for growth of the implanted tumor. At 14 days post-implantation, rats were enrolled in the Electro-NT study. Rat 1 served as the control rat and received an IV injection of 0.2 mL SPIOs at a concentration of 5 mg of iron/ml. The SPIOs in the example were 15 nm in diameter and were functionalized with doxorubicin. The control rat 1 was imaged using a T2-weighted Turbo Spin Echo (T2W-TSE) MR sequence following nanoparticle administration to confirm delivery. The control rat (Rat 1) was kept alive for 100 minutes, sacrificed and necropsied to obtain tissue samples. Rat 2 served as a treatment rat. Rat 2 received the same IV injection of nanoparticles as Rat 1, which was then followed by EP. To perform EP, the liver of Rat 2 was exposed surgically through a mini-laparotomy. EP electrodes were placed into the tumor and an electric field was applied using the following parameters: 1300 V, 8 pulses, 100 microseconds pulse time, at 100 millisecond intervals. Rat 2 was also kept alive for 100 minutes then sacrificed and necropsied for tissue harvest. Samples of tumor tissue were quantitatively analyzed for iron (Fe) content using inductively coupled plasma mass spectroscopy. The example results are shown in the following table:

| | Nanograms of Fe per | | Ratio |
|---|---|---|---|
| | gram of tissue | | Electro- |
| Tissue | Electro-NT | control | NT/control |
| N1S1 Tumor Core | 81.068 | 14.5580 | 5.5686 |
| N1S1 Tumor | 53.957 | 21.335 | 2.5290 |
| Periphery | | | |

As shown by the example results, the amount of SPIOs taken up or absorbed by the tumor tissue using Electro-NT was greater than the amount delivered to the tumor using IV administration alone—with increased penetration of the particles into the tumor core. These findings are also observed using MR imaging, which shown increased tissue contrast within an electroporated tumor zone compared to the control tumor.

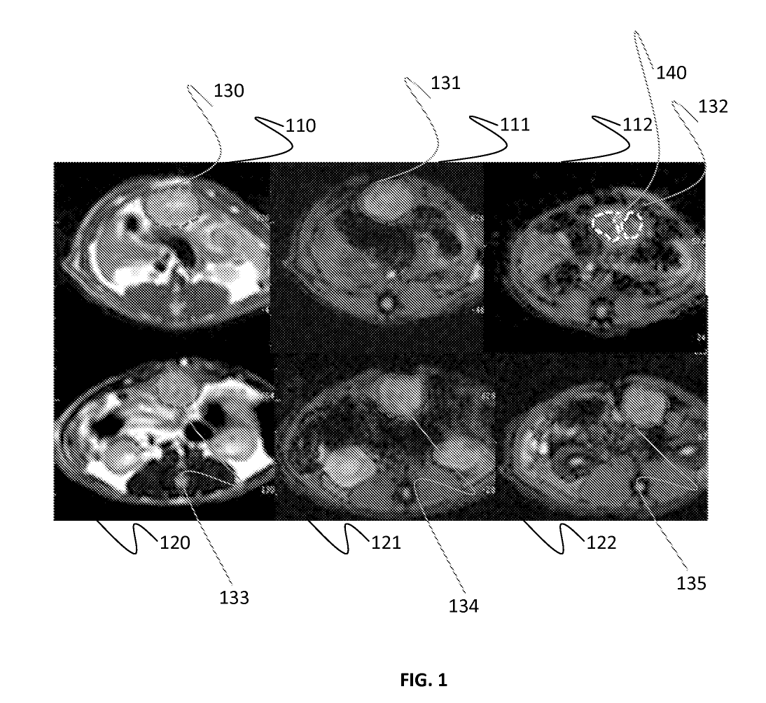

FIG. 1 illustrates example T2W-TSE and gradient T2*-weighted recalled echo (T2*W-GRE) (TE: 31.5 ms) MRI images from an example N1S1 rat model. A series of images 110-112 depicts NP delivery with electroporation. A series of images 120-122 depicts NP delivery without electroporation. As shown in images 110-112 and 120-122, a tumor can be identified in the image 110-112, 120-122 using a visual indicator 130-135 (e.g., circling or highlighting in a color such as red). As depicted in image 112, nanoparticles are identified with a second visual indicator 140 (e.g., circled or highlighted in a second color different from the first color, such as white). Note that delivery with electroporation significantly increased tumor nanoparticle uptake compared to delivery without electroporation.

In certain examples, an NP therapeutic can be scaled-up and produced in bulk for sale. An NP therapeutic material can be adapted or configured to treat a variety of solid tumor malignancy(ies), cause tissue growth in a solid organ requiring it (e.g., liver regeneration, heart regeneration after a myocardial infarction, etc.), deliver molecular imaging agent(s) to tissue(s) of interest, etc. Coupled with EP, NPs can be guided specifically to target tissue(s) with high resulting uptake or absorption in the tissue(s). Electro-nanotherapy can be developed to treat and/or diagnose a variety of disease processes including cancers, solid organ disease, cardiac disease, etc., using efficient delivery of NPs to desired site(s) in a body.

As discussed above, electroporation (EP) can be utilized to modulate influx of chemotherapeutics into tumor cells, both in vitro and in vivo. During EP, when cells are exposed to brief direct current, the electric field induces transient cell membrane channels, which form temporary pores. The temporary pores allow passage of extracellular macromolecules into the cytosol of the cell. The electric pulses also induce transient vascular hypoperfusion within the treated zone. A resultant restriction of flow diminishes washout of therapeutics from the treated zone. In certain examples, the therapeutic agent has already been administered to the patient and is already within the target EP zone at the time of treatment. In other examples, the therapeutic agent is administered to the patient at the time of EP treatment and/or shortly following EP treatment with the treated zone still exhibits greater permeability.

To explore the relationship between the timing of therapeutic delivery and tumor EP, therapeutic superparamagnetic iron oxide nanoparticles (SPIOs) were utilized in an example to serve as a dual MR imaging agent and drug delivery vehicle. Eight VX2 tumors were surgically implanted in rabbit hind limbs. SPIO-NPs were obtained and functionalized with doxorubicin (e.g., mean diameter=10 nm). In the example, all animals underwent anatomic T2 turbo spin echo (TSE) imaging to confirm tumor growth and location, and T2 weighted (T2*W) imaging to determine baseline tumor signal intensity. Following scans, animals were transferred to the angiography suite for carotid artery catheterization and femoral artery angiography under X-Ray Digital Subtraction Angiography guidance to confirm ideal catheter placement prior to therapeutic delivery (e.g., SPIO+ethiodol). Each tumor was then electroporated at different times relative to SPIO embolization (e.g., range: −5 minutes to +3 minutes). T2*W images were then obtained post-procedurally to confirm NP delivery and evaluate tumor signal changes. The rabbits were then euthanized and tissues were harvested to determine SPIO uptake using inductively coupled plasma mass spectroscopy (ICP-MS). Mean SPIO-NP concentration within all tumors were compared between timing groups using ANOVA with post-hoc Tukey analysis. A p<0.05 was considered significant.

In the example, ICP-MS analysis of iron content demonstrated that tumors that underwent EP for 1.5 to 2.25 minutes following injection showed a 2.9 fold increase in SPIO concentration compared to all other time points (p<0.05). These findings were confirmed by a noted decrease in MR signal intensity on T2*W imaging. Groups that underwent EP outside this window did not demonstrate appreciable T2*W signal changes within their tumors.

The timing of EP, relative to intra-arterial (IA) therapeutic embolization, for example, can affect tumor uptake of the NPs. In the example, a therapeutic window was observed to occur between 1.5 to 2.25 minutes following therapeutic delivery, for example. A decrease in uptake was noted when EP occurred outside this window. Furthermore, these findings can be observed non-invasively using T2*W imaging in vivo. EP can be shown to provide efficacy in tumor therapy using these timing parameters.

Hepatocellular carcinoma (HCC) is the sixth most common cancer worldwide and the third most common cause of cancer death. New therapies can help interventional radiologists to improve patient outcomes. As discussed above, nanoparticles represent a promising new drug delivery platform. However, systemic administration results in sequestration by the healthy liver tissue, and minimal tumor uptake. Using an N1-S1 rat model, reversible electroporation (EP) was tested in an example to determine an increase intratumoral uptake of therapeutic superparamagnetic iron oxide (SPIO) nanoparticles loaded with doxorubicin (DOX).

In the example, using the N1-S1 rodent model, hepatomas were grown in twelve Sprague-Dawley rats that were divided into treatment and control groups. Magnetic resonance (MR) imaging was performed at 10-14 days to confirm tumor growth. For both groups, 0.56 mg/kg body weight SPIO-DOX nanoparticles were injected via the femoral vein. For the treatment group, EP electrodes were inserted and 8 pulses (e.g., 100-ns pulse duration, 1,300-V/cm field strength) were applied to liver tumors 1.5 minutes post-SPIO-DOX injection. T2*-weighted imaging was performed on both groups to visualize nanoparticle delivery and uptake. Both groups were sacrificed and tumors were harvested for evaluation by ICP-MS for iron concentration. Prussian Blue staining was done to visualize iron content. Iron concentrations between the groups were compared with paired t-tests, with p<0.5 considered significant.

In the example, N1S1 tumors were grown in all twelve rats. Electroporation resulted in increased uptake within the tumor tissue over IV delivery alone. Within the tumor core a 5.57 times or 6.3 times, for example, increase in iron content over IV delivery was observed (p<0.05). In an example, these findings were also confined on T2*W MRI in vivo and Prussian Blue staining of tumor specimens.

Thus, EP enhances tumor uptake of SPIO-DOX nanoparticles and can serve to improve tumor uptake of other therapeutic nanoparticles.

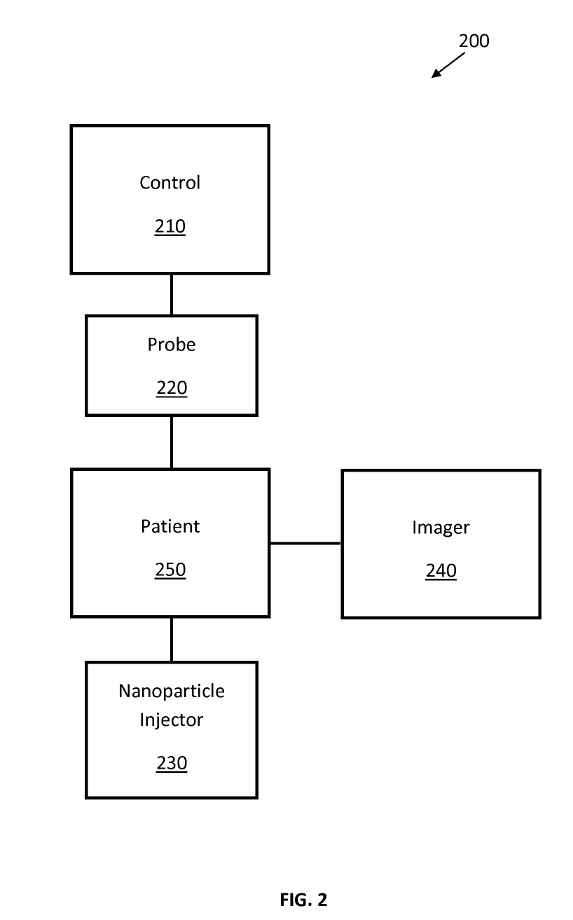

FIG. 2 illustrates an example system 200 for cell electro-nanotherapy. The illustrated system 200 includes a controller 210, an electric probe 220, a nanoparticle injector 230, and an imager 240 arranged and operating with respect to a patient 250.

In the illustrated example, the controller 210, such as an AngioDynamics HPV01 Generator, supplies a series of one or more electrical pulses to the probe 220. A user and/or program setting can specify one or more characteristics of the series of pulses using the controller 210. The controller 210 can be used to set a voltage, number of pulses, pulse duration, and/or pulse interval, etc. The configured series of pulses is triggered by the controller 210 for generation through the probe 220 (e.g., an AngioDynamics NanoKnife™ needle and/or other single or multiple electrical probes).

In the illustrated example, the controller 210 includes a function generator. One or more electrodes forming the probe 220 are attached to the function generator to apply a voltage at a target site with respect to the patient 250. Parameters used to configure the probe 220 for electroporation can include, for example, a voltage between 250-1500 Volts per centimeters (V/cm); a number of pulses between 4 and 10; a pulse duration between 99 microseconds (μs) to 100 milliseconds (ms); a pulse interval between 100 ms and 1 Hertz (Hz); and a number of electrodes between 1 and 8.

The probe 220 of the illustrated example is positioned with respect to a cell site of interest on and/or in the patient 250. For example, the probe 220 can be positioned on the skin of the patient 250 against and/or over the cell site of interest (e.g., cutaneously). Alternatively or in addition, the probe 220 can be inserted into the patient 250 to be adjacent to the cell site of interest within the patient 250 (e.g., inserting a needle probe 220 near an organ and/or other tumor site of interest), for example. Thus, the electroporation device 220 can be placed through the skin (percutaneously) or via surgical laparoscopy to access the tissue site. In some examples, a combination of surgical, percutaneous, and/or cutaneous probe(s) 220 can be used to facilitate electroporation.

The nanoparticle injector 230 of the illustrated example provides nanoparticles to the patient 250 (e.g., to a particular tissue site of interest in the patient 250) via systemic and/or local delivery. In the example of FIG. 2, introduction of nanoparticles into the patient 250 is timed with respect to the series of pulses. For example, the injection can occur 1.5 minutes before the series of pulses. The nanoparticle injector 230 can include an intravenous (IV) catheter, a catheter placed in the arterial blood supply of the tumor (e.g., electroporation embolization), etc. Electroporation embolization, for example, can provide better delivery of local nano-therapeutics. Embolic agents can slow blood flow and increase the dwell time of the nanoparticles at the cell site, for example. The nanoparticle injector 230 can also include a needle to be inserted into a tumor and used to directly inject the nanoparticles. In some examples, the probe 220 and the injector 230 can be integrated to provide a needle electrode to deliver/inject nanoparticles and provide electrical pulses. In another example, at the time of surgery, a tumor can be bathed directly with the therapeutic nanoparticles.

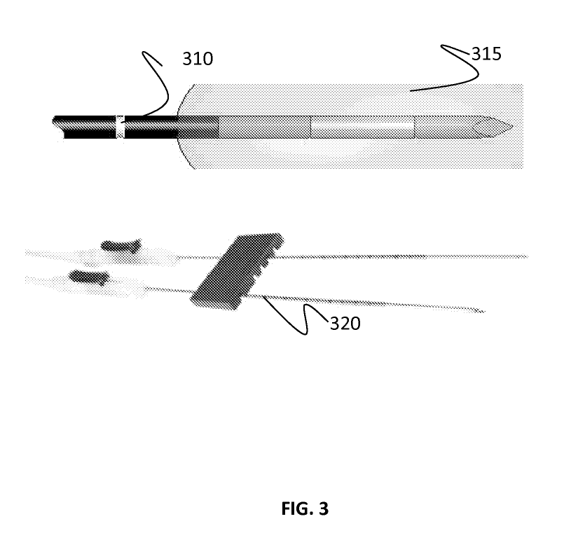

Some examples of needle electrode probes are shown in FIG. 3. FIG. 3 illustrates an example single needle electrode probe 310 inserted at a target tissue site 315. FIG. 3 also illustrates a double need electrode probe 320 (e.g., the AngioDynamics NanoKnife™).

After nanoparticles have been introduced in the patient 250 via the injector 230 and a series of pulses have been applied at a patient cell site of interest by the probe 220 to increase permeability for the nanoparticles at the cell site, results can be evaluating using the example imager 240 of FIG. 2. For example, electro-nanotherapy results can be verified using an MRI. Alternatively or in addition, results can be verified using a mass spectrometry analysis (e.g., inductively coupled plasma (ICP) mass spectrometry) of the uptake of the nano-agent based on an obtained tissue sample. The imager 240 can additionally or alternatively be used to visualize electrode placement and determine other positioning information for image-guided surgery, image-guided delivery of nanoparticles, etc.

While an example manner of implementing an electro-nanotherapy has been illustrated in FIG. 2, one or more of the elements, processes and/or devices illustrated in FIG. 2 can be combined, divided, re-arranged, omitted, eliminated and/or implemented in any other way. Further, the example controller 210, the example probe 220, the example injector 230, the example imager 240, and/or, more generally, the example system 200 of FIG. 2 can be implemented by hardware, software, firmware and/or any combination of hardware, software and/or firmware. Thus, for example, any of the example controller 210, the example probe 220, the example injector 230, the example imager 240, and/or, more generally, the example system 200 of FIG. 2 could be implemented by or include one or more circuit(s), programmable processor(s), application specific integrated circuit(s) (ASIC(s)), programmable logic device(s) (PLD(s)) and/or field programmable logic device(s) (FPLD(s)), etc. When any of the appended apparatus claims are read to cover a purely software and/or firmware implementation, at least one of the example controller 210, the example probe 220, the example injector 230, or the example imager 240 is hereby expressly defined to include a computer readable medium such as a memory, DVD, CD, Blu-ray, etc., storing the software and/or firmware. Further still, the example system 200 of FIG. 2 can include one or more elements, processes and/or devices in addition to, or instead of, those illustrated in FIG. 2, and/or can include more than one of any or all of the illustrated elements, processes and devices.

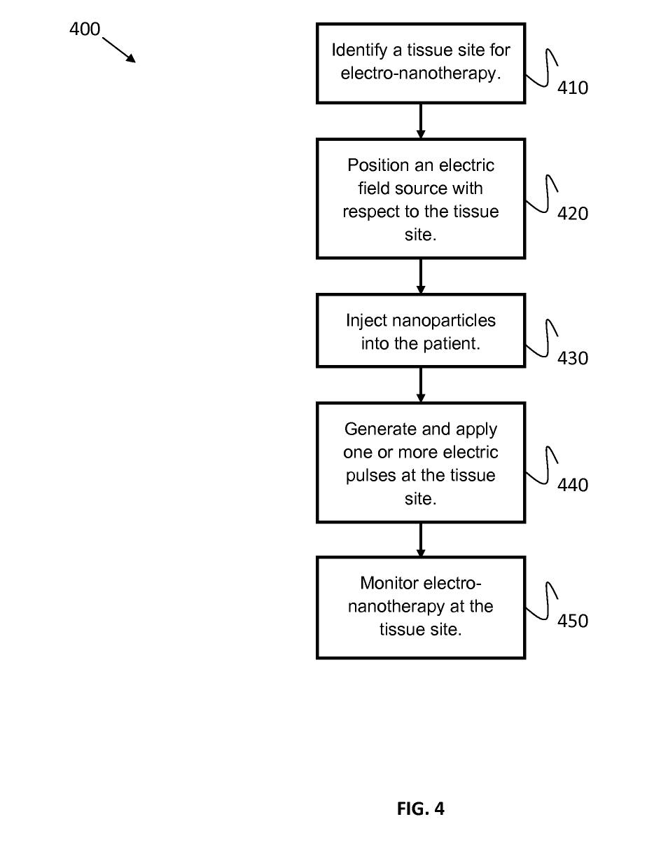

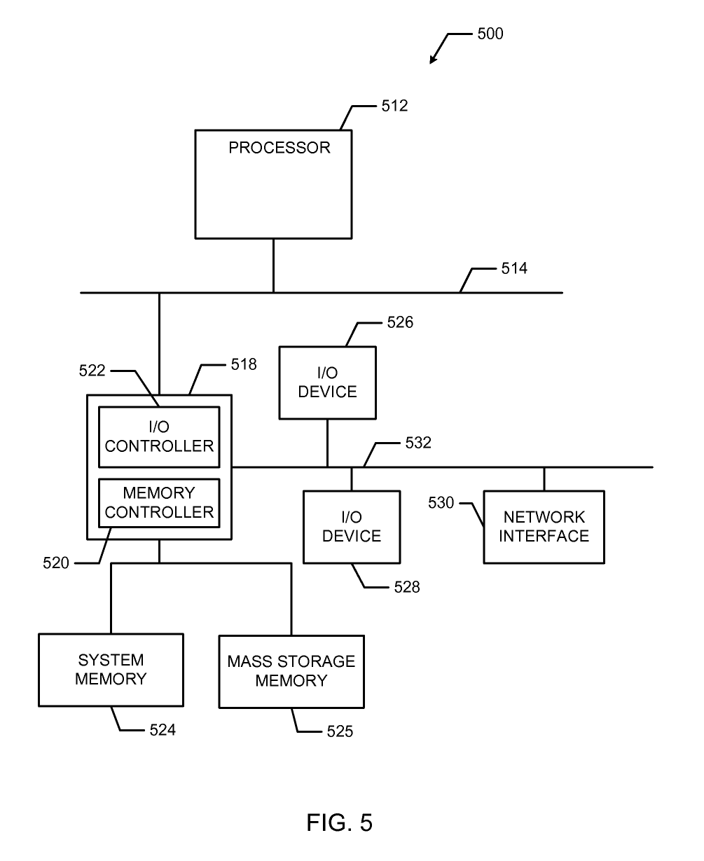

A flowchart including blocks representative of example machine readable instructions for implementing some or all of the system 200 of FIG. 2 is shown in FIG. 4. In this example, the machine readable instructions include a program for execution by a processor such as the processor 512 shown in the example computer 500 discussed below in connection with FIG. 5. The program can be embodied in software stored on a computer readable medium such as a CD-ROM, a floppy disk, a hard drive, a digital versatile disk (DVD), or a memory associated with the processor 512, but the entire program and/or parts thereof could alternatively be executed by a device other than the processor 512 and/or embodied in firmware or dedicated hardware. Further, although the example program is described with reference to the flowchart illustrated in FIG. 4, many other methods of implementing the example system 200 (and/or one or more portions of the system 200) can alternatively be used. For example, the order of execution of the blocks can be changed, and/or some of the blocks described can be changed, eliminated, or combined. Additionally or alternatively, some or all of the method of FIG. 4 can be performed manually by, for example, a surgeon.

As mentioned above, the example processes of FIG. 4 can be implemented using coded instructions (e.g., computer readable instructions) stored on a tangible computer readable medium such as a hard disk drive, a flash memory, a read-only memory (ROM), a compact disk (CD), a digital versatile disk (DVD), a cache, a random-access memory (RAM) and/or any other storage media in which information is stored for any duration (e.g., for extended time periods, permanently, brief instances, for temporarily buffering, and/or for caching of the information). As used herein, the term tangible computer readable medium is expressly defined to include any type of computer readable storage and to exclude propagating signals. Additionally or alternatively, the example processes of FIG. 4 can be implemented using coded instructions (e.g., computer readable instructions) stored on a non-transitory computer readable medium such as a hard disk drive, a flash memory, a read-only memory, a compact disk, a digital versatile disk, a cache, a random-access memory and/or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

FIG. 4 depicts a flow diagram for an example method 400 for electro-nanotherapy of a patient. At block 410, a tissue site for electro-nanotherapy is identified. For example, a tumor site or a tissue site adjacent to a tumor is identified. The site can be identified via image (e.g., an MRI image), biopsy, surgical incision, etc.

At block 420, positioning of an electric field source (e.g., a needle or probe electrode) is performed and/or enabled to be performed with respect to the tissue site. For example, a user, such as a surgeon and/or other clinician, can position the electric field source on and/or in the patient with respect to the tissue site the user wishes to have increased permeability. The electric field source can include and/or be connected to a controller including a user interface and a power unit. The user interface accepts user input and calculates treatment parameters based on the input and possibly other stored information. The power unit generates electrical pulses based on the treatment parameters. Using the controller, timing delays and triggering signals can be configured and provided to the electric field source.

At block 430, nanoparticles are injected and/or injection is facilitated into the patient. Injection can be performed using one or more devices include IV injection, IA injection, surgical introduction of nanoparticles, bathing of a tumor and/or other cell site in NPs, etc.

At block 440, a series of one or more electric pulses is generated and applied at the tissue site. Electroporation can employ micro to millisecond electric pulses to create pores in the cell membrane, thus allowing molecules that, due to their physical and/or chemical properties, would normally not be able to cross the cell membrane, to enter the cell. Using a control, electroporation can be triggering using a series of electrical pulses generated at a needle and/or other probe using a power supply in the control. In electrochemotherapy, a combination of chemotherapy and electroporation of tumors, the effects of nanoparticles or drugs are increased. The opening of pores in the cell membrane allows a chemotherapeutic agent to enter the cell at greater, more effective concentration. An electroporator device can use one or more electrodes to apply an electric field with a desired appropriate shape and intensity to homogenously cover the target tissue. A switching unit can be used with the controller to route treatment to the electrodes in sequenced fashion.

In some examples, nanoparticles are injected prior to generation and application of electric pulses. In some examples, nanoparticles are injected while and/or after electric pulses are applied to the tissue site.

At block 450, electro-nanotherapy at the tissue site is monitored. For example, one or more images, such as MRI images, can be obtained to monitor and review results of the electro-nanotherapy. Alternatively or in addition, results can be verified using a mass spectrometry analysis of a tissue sample to determine absorption of the nanoparticles. Monitoring can also include images taken to visualize electrode placement and determine other positioning information for image-guided surgery, image-guided delivery of nanoparticles, etc., before, during, and/or after nanoparticle insertion and/or electrical pulse generation, for example.

FIG. 5 is a block diagram of an example computer or other processor system 500 that can be used to execute one or more of the blocks of FIG. 4 to implement systems, apparatus, and methods described herein, including the controller, probe, injector, and/or imager of FIG. 2. For example, the system 500 can be used to implement the controller and provide control of the probe, injector, and/or imager of FIG. 2. As shown in FIG. 5, the processor system 500 includes a processor 512 that is coupled to an interconnection bus 514. The processor 512 can be any suitable processor, processing unit, or microprocessor, for example. Although not shown in FIG. 5, the system 500 can be a multi-processor system and, thus, can include one or more additional processors that are identical or similar to the processor 512 and that are communicatively coupled to the interconnection bus 514.

The processor 512 of FIG. 5 is coupled to a chipset 518, which includes a memory controller 520 and an input/output (“I/O”) controller 522. As is well known, a chipset typically provides I/O and memory management functions as well as a plurality of general purpose and/or special purpose registers, timers, etc. that are accessible or used by one or more processors coupled to the chipset 518. The memory controller 520 performs functions that enable the processor 512 (or processors if there are multiple processors) to access a system memory 524 and a mass storage memory 525.

The system memory 524 can include any desired type of volatile and/or non-volatile memory such as, for example, static random access memory (SRAM), dynamic random access memory (DRAM), flash memory, read-only memory (ROM), etc. The mass storage memory 525 can include any desired type of mass storage device including hard disk drives, optical drives, tape storage devices, etc.

The I/O controller 522 performs functions that enable the processor 512 to communicate with peripheral input/output (“I/O”) devices 526 and 528 and a network interface 530 via an I/O bus 532. The I/O devices 526 and 528 can be any desired type of I/O device such as, for example, a keyboard, a video display or monitor, a mouse, etc. The network interface 530 can be, for example, an Ethernet device, an asynchronous transfer mode (“ATM”) device, an 802.11 device, a DSL modem, a cable modem, a cellular modem, etc. that enables the processor system 500 to communicate with another processor system.

While the memory controller 520 and the I/O controller 522 are depicted in FIG. 5 as separate blocks within the chipset 518, the functions performed by these blocks can be integrated within a single semiconductor circuit or can be implemented using two or more separate integrated circuits. The coded instructions of FIG. 4 can be stored in the mass storage device 525, in the system memory 524, and/or on a removable storage medium such as a CD, Blu-ray, or DVD.

From the foregoing, it will appreciate that methods, apparatus, and articles of manufacture have been described which improve cell permeability and/or delivery of nanoparticles to a target tissue site for cell treatment.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. A method for electro-nanotherapy of cells at a tissue site, the method comprising:
facilitating identification of a tissue site for electro-nanotherapy;
facilitating injection of nanoparticles at the tissue site; and
enabling generation of one or more electric pulses at the tissue site to create pores in a cell membrane at the tissue site such that the nanoparticles penetrate the cell membrane.

2. The method of claim 1, further comprising monitoring the tissue site to verify penetration of the cell membrane by the nanoparticles.

3. The method of claim 2, wherein monitoring comprises monitoring via at least one of magnetic resonance images and a mass spectrometry analysis.

4. The method of claim 1, wherein the nanoparticles comprise superparamagnetic iron oxide nanoparticles treated with a chemotherapeutic.

5. The method of claim 5, wherein the chemotherapeutic comprises doxorubicin.

6. A superparamagnetic iron oxide nanoparticle treated with a chemotherapeutic to be absorbed by a cell membrane in patient tissue when the tissue is stimulated using a series of electrical pulses, the nanoparticle adapted to be imaged to permit image-guided drug delivery.

7. The nanoparticle of claim 6, wherein the chemotherapeutic comprises doxorubicin.

8. The nanoparticle of claim 6, wherein the chemotherapeutic is coated on the nanoparticle by covalently attaching the chemotherapeutic to the nanoparticle.

9. The nanoparticle of claim 6, wherein the nanoparticle has a size of between one and one hundred nanometers in diameter.

10. A system for electro-nanotherapy of cells at a tissue site in a patient, the system comprising:
a nanoparticle injector to facilitate injection of nanoparticles treated with a chemotherapeutic at an identified tissue site; and
a controller to enable generation of one or more electrical pulses at the tissue site to create pores in a cell membrane at the tissue site such that the nanoparticles penetrate the cell membrane.

11. The system of claim 10, further comprising an imager to monitor the tissue site to verify penetration of the cell membrane by the nanoparticles.

12. The system of claim 11, wherein the imager is to monitor via at least one of magnetic resonance images and a mass spectrometry analysis.

13. The system of claim 10, wherein the nanoparticles comprise superparamagnetic iron oxide nanoparticles treated with a chemotherapeutic.

14. The system of claim 13, wherein the chemotherapeutic comprises doxorubicin.

15. The system of claim 13, wherein the chemotherapeutic is coated on the nanoparticle by covalently attaching the chemotherapeutic to the nanoparticle.

16. The system of claim 13, wherein the nanoparticle has a size of between one and one hundred nanometers in diameter.

17. The system of claim 10, wherein the nanoparticle injector comprises at least one of a tube and a needle for delivery of the nanoparticles at the tissue site.

18. The system of claim 10, wherein the controller is to set one or more characteristics of the series of electrical pulses.

19. The system of claim 10, wherein the controller is attached to a probe to deliver the series of electrical pulses to the tissue site.

20. The system of claim 19, further comprising a switching unit to route treatment in a sequence to one or more electrodes in the probe.
